(19) United States
(12) Patent Application Publication
(10) Pub. No.: US 2005/0152842 A1
(43) Pub. Date: Jul. 14, 2005

(54) Poly (L-glutamic acid) paramagnetic material complex and use as a biodegradable MRI contrast agent

(76) Inventors: Chun Li, Missouri City, TX (US); Xiaoxia Wen, Manvel, TX (US); Edward F. Jackson, Pearland, TX (US); Sidney Wallace, Bellaire, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/021,879

(22) Filed: Dec. 22, 2004

Related U.S. Application Data
(60) Provisional application No. 60/532,555, filed on Dec. 24, 2003.

(51) Int. Cl. A61K 49/00

(52) U.S. Cl. 424/9.322

(57) ABSTRACT

The present invention includes PG polymer complexes with paramagnetic materials, such as Gd, Mn and iron oxide. The complexes may also include chelating agents which may be covalently attached to the PG polymer backbone through linkers. PEG may also be attached to the PG polymer backbone. The complexes may include targeting molecules. The complexes are useful as MRI contrast agents, particularly as blood pool agents.

### PRIORITY CLAIM

The present application claims priority under 35 U.S.C. §119(e) to U.S. Provisional Patent Application 60/532,555, filed Dec. 24, 2003, titled “Poly (L-Glutamic Acid) Paramagnetic Material Complex and Use as Biodegradable MRI Contrast Agent”.

### STATEMENT OF GOVERNMENT INTEREST

Portions of the present invention were developed using funding provided by the National Institutes of Health, Contract Number U54 CA90810. The U.S. Government has certain rights in the invention.

### FIELD OF THE INVENTION

The present invention relates in general to paramagnetic material and polymer complexes. Additionally, the invention relates to magnetic resonance imaging (MRI) and in specific to use of paramagnetic material and polymer complexes as MRI contrast agents for blood pool imaging that has more prolonged contrast enhancement of the vasculatures-arteries, venin, inflammation, infection, tumor, etc. than Gd-DTPA.

### BACKGROUND OF THE INVENTION

Medical diagnostic imaging is achieved through three principle mechanisms: the emission or absorption of radiation, nuclear magnetic moments and relaxation, and transmission or reflection of ultrasound. Magnetic resonance imaging (MRI) utilizes the transition between different “spin” states in atomic nuclei under the action of a radio frequency signal to produce an image. This relates to the magnetic properties of the material being imaged. Therefore, paramagnetic materials, which exhibit some magnetic properties, are often easy to detect using MRI.

Whatever imaging modality is used, medical imaging requires that the intensity of a signal from an area of interest be sufficient to differentiate the area from surrounding tissues. In many cases this cannot be achieved without the use of contrast agents because non-enhanced imaging techniques are useful only when relatively large tissue areas are involved in a disease process. To achieve a sufficient differentiation of a pathologic lesion from surrounding tissue, contrast agents are used to absorb certain types of signal to a greater degree than surrounding tissue. For example, for MRI, a paramagnetic ion such as gadolinium (Gd), Manganese (Mn) or iron oxide might be used in a contrast agent composition to intensify the magnetic signal of the area of interest.

Imaging of vascular structures is of critical importance in clinical medicine. Many diseases, e.g. infection, abnormal blood vessel growth or vascular occlusion through thrombosis or atherosclerotic lesions may produce abnormal images. Existing techniques to image blood vessels include angiography which is an invasive technique that may be painful, expensive and of increased risk to a patient than a noninvasive imaging technique. However, in order to provide sufficient detail of vascular structures and function, a noninvasive imaging technique, such as magnetic resonance imaging (MRI) is usually used in combination with the intravenous administration of a contrast agent that concentrates in the blood vessels (commonly referred to as a “blood pool agent”). Blood pool agents, therefore, enhance the anatomic detail of blood vessels imaged by MRI.

Blood pool agents are also used with MRI to estimate the degree of new microvasculature, known as “angiogenesis” that occurs as a tumor grows. Angiogenesis is a central process in tumor growth and in the potential for a tumor to metastasize. All solid tumors require angiogenesis for growth and metastasis and the degree of angiogenesis is one measure of a tumor's growth rate and aggressive nature. It is therefore of clinical importance to monitor angiogenesis in tumors in a noninvasive manner such as with MRI.

Ideally, an MRI contrast agent resides in the blood long enough to allow for thorough MRI examinations, then is degraded and cleared from the body after completion of the imaging procedures. An MRI blood pool contrast agent also preferably has high relaxivity to increase the intensity of the tissue signal. Relaxivity results from the interaction of the contrast agent and water and relates to the speed with which detectable changes in spin states in the atomic nuclei occur. In compounds with high relaxivity, spin state changes occur often, allowing for an increased number of detectable spin changes within a given time period.

In addition to high relaxivity, a contrast agent also preferably exhibits slow clearance by the kidneys, thereby prolonging the circulation time of the contrast agent. Gd and Mn provide sufficient changes in relaxivity for MRI, however, because of toxicity and poor solubility of free paramagnetic heavy metal cations at physiologic pH, chelated complexes are used to decrease toxicity. Existing formulations of chelated Gd are not suitable for use as contrast agents for blood pool imaging due to their partial leakage through the vessel walls (extravasation into the extravascular space), their rapid elimination through the kidneys, or their general toxicity, among other problems.

Although polymers have previously been combined with paramagnetic materials in MRI contrast agents, many polymers suffer from a variety of problems when used in this manner. For example, paramagnetic materials have been conjugated to poly(L-lysine), which has negative biological effects due to the presence of multiple positive charges. Dextran, dendrimers and polyamides have also been used as polymeric blood-pool contrast agents but these materials are not biodegradable, leaving ample room for improvement.

The spread of cancer cells to regional lymph nodes is the most reliable predictor of adverse outcomes in many solid tumors. However, early spread of tumors to lymph nodes may evade detection because traditional clinical evaluation of lymph node metastases are not very sensitive, and therefore patients often undergo unnecessary removal of entire regional nodal basins for precautionary pathological assessment. Clinical evaluation of lymphatic metastasis has improved recently with lymphatic mapping with sentinel node biopsy (LSNB), a procedure that improves the accuracy of the evaluation while avoiding unnecessary surgical resection. However, LSNB remains an invasive procedure, the results of which may not be known for up to two weeks. A minimally invasive method for identifying sentinel lymph nodes and quickly assessing tumor invasion will have significant impact on both therapy and prognosis.

Methods have been proposed for non-invasive evaluation of lymph node metastases based on the relative accumulation of contrast agent being either promoted or prevented by the presence of tumor tissue. Primary requirements for contrast-enhanced identification of sentinal lymph nodes are that the agent be small enough to penetrate beyond the vascular bed, yet large enough to be retained within the lymphatic system, while providing adequate signal enhancement in concentrations that can accumulate relatively quickly.

### SUMMARY OF THE INVENTION

The following abbreviations are used throughout the specification:

6-aminohexyl PG—a modified PG of the general formula PG[-NH(CH₂)₆NH₂]ₙ, where n is greater or equal to 1.
BZDTPA—benzyl DTPA
DTPA—diethylenetriamine pentaacetic acid
DOTA—1,4,7,10-tetraazacyclododecane-N,N′,N″,N″-tetraacetic acid
Gd—Gadolinium
Mn—Manganese
PEG—polyethylene glycol
PG—poly(L-glutamic acid) or poly(L-glutamine)
PG-DTPA-Gd—a polymer complex containing a PG polymer with DTPA-Gd covalently attached to one or more PG side chains.
PG/PEG-DTPA-Gd—a polymer complex containing a PG polymer with DTPA-Gd covalently attached to one or more PG side chains and with PEG covalently attached to one or more different PG side chains.
PG-BzDTPA-Gd—a polymer complex of the general formula PG-[BzDTPA-Gd]ₙ, where n is greater than or equal to 1.
PEG/PG-BzDPTA-Gd—a polymer complex of the general formula PEG-grafted-PG-[BzDTPA-Gd]ₙ, where n is greater than or equal to 1.

PG-Hex-BzDTPA-Gd—a polymer complex of the general formula PG[-NH(CH₂)₆NHC(S)NH-BzDTPA-Gd]ₙ, where n is greater than or equal to 1.
PEG/PG-Hex-BzDTPA-Gd—a polymer complex of the general formula PEG-grafted-PG[-NH(CH₂)₆NHC(S)NH-BzDTPA-Gd]ₙ, where n is greater than or equal to 1
PG-Hex-DTPA-Gd—a polymer complex of the general formula PG[-NH(CH₂)₆NH-DTPA-Gd]ₙ, where n is greater than or equal to 1.
PEG/PG-Hex-DTPA-Gd—a polymer complex of the general formula PEG-grafted-PG[-NH(CH₂)₆NH-DTPA-Gd]ₙ, where n is greater than or equal to 1.

One embodiment of the invention relates to a PG polymer complex including a PG polymer, at least one DTPA molecule covalently bound to the side chain carboxyl group of the PG polymer and at least one Gd, Mn or iron oxide ion chelated to the DTPA.

The number average molecular weight of the PG polymer complex may be between approximately 10,000 and 500,000. In more specific embodiments, it may be between approximately 50,000 and 300,000.

In other embodiments, the DTPA molecule may be covalently bound to the PG polymer through a linker. More specifically, this linker may be a hexane diamine or a PEG polymer of the formula —(CH₂CH₂O)ₙ— where n=1 to 40.

The invention may include at least one PEG polymer covalently bound to the side chain carboxyl groups of the PG polymer. This PEG polymer may have a molecular weight of at least 3,000.

Complexes of the present invention may also include a targeting molecule covalently attached to the complex.

Another embodiment of the invention relates to a complex including a PG polymer and a paramagnetic material attached to the PG polymer.

The complex may further include a chelating agent, covalently bound to the PG polymer which chelates the paramagnetic material. The paramagnetic material may include Gd, Mn or iron oxide. The number average molecular weight of the complex may be between approximately 10,000 and 500,000 Another embodiment of the present invention relates to an MRI contrast agent including a molecule of the formula: 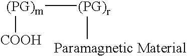
where r is at least one.

A different embodiment relates to another MRI contrast agent including a molecule of the formula: 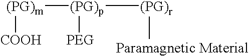
where p is at least one and r is at least one.

The invention also includes a method of making a PG polymer complex by modifying at least one side chain carboxyl group of a PG polymer, attaching at least one chelating agent molecule to a modified side chain carboxyl group and chelating a paramagnetic material.

In the method, at least 5% of all available side chain carboxyl groups of the PG polymer may be modified. Additionally, a chelating agent molecule may be attached to at least 5% of all available modified side chain carboxyl groups.

The chelating agent may be DTPA or DOTA and the paramagnetic material may include Gd, Mn or iron oxide.

The method may additionally include attaching at least one PEG polymer to a modified side chain carboxyl group and/or attaching at least one targeting molecule to the PG polymer.

Another embodiment of the present invention relates to a method of making an MRI contrast agent by adding t-Boc-NH(CH₂)₆NH₂/DCC to a solution of PG polymer to produce a first product, adding trifluoroacetic acid to the first product to produce a second product, adding DTPA-dianhydride to the second product to produce a third product, and adding GdCl₃ to the third product to produce a fourth product.

Another method of making an MRI contrast agent includes adding t-Boc-NH(CH₂)₆NH₂/DCC to a solution of PG polymer to produce a first product, adding trifluoroacetic acid to the first product to produce a second product, adding mPEG-NHS to the second product to produce a third product, adding DTPA-dianhydride to the third product to produce a fourth product, and adding GdCl₃ to the fourth product to produce a fifth product.

Yet another method of making a MRI contrast agent includes adding NHS/DCC to a solution of PG to produce a first product, adding SCN-Bz-DTPA to t-Boc-NH(CH₂)₆NH₂ to produce a second product, adding trifluoroacetic acid to the second product to produce a third product, adding the first product to the third product to produce a fourth product, and adding GdCl₃ to the fourth product to produce a fifth product.

An additional method of making a MRI contrast agent includes adding NHS/DCC to a solution of PG to produce a first product, adding SCN-Bz-DTPA to t-Boc-NH(CH₂)₆NH₂ to produce a second product, adding trifluoroacetic acid to the second product to produce a third product, adding methoxyl-PEG-NH₂ to the first product to produce the fourth product, adding the fourth product to the third product to produce a fifth product, and adding GdCl₃ to the fifth product to produce a sixth product.

Finally, another method of making a MRI contrast agent includes adding p-NH₂-Bz-DTPA-penta(t-butyl ester) to PG in diisopropylcarbodiimine to produce a first product. Trifluoroacetic acid is then added to the first product to produce a second product. Finally, GdCl₃ is added to the second product to product PG-BzDTPA-Gd.

Complexes described above and produced by the methods set forth above may be used for a variety of purposes. Accordingly, these complexes may be used in methods of detecting a vasculature leakage, an ulcer, inflammation, angiogenesis, cancer metastases and vascular occlusion such as that resulting from thrombosis or an atherosclerotic lesion by providing such a complex to a patient. Additionally, the complexes may be used as contrast agents in conduction angiography.

In specific embodiments, the complexes may be used to image necrosis and enzyme activity or to map lymphatic drain.

### BRIEF DESCRIPTION OF THE DRAWINGS

A more complete and thorough understanding of the present embodiments and advantages thereof may be acquired by referring to the following description taken in conjunction with the accompanying drawings.

FIG. 1 illustrates a method of forming a PG-Hex-DTPA-Gd complex according to an embodiment of the present invention.

FIG. 2 illustrates a method of forming a PEG/PG-Hex-DTPA-Gd complex according to an embodiment of the present invention.

FIG. 3 is a gel permeation chromatography (GPC) chromatogram of a PG-Hex-DTPA-Gd complex according to an embodiment of the present invention showing retention time (in minutes) versus normalized light signal intensity. The solid line represents light scattering, while the dashed line represents refractive index. The GPC was performed on a TSK-G3000PW column eluted with PBS containing 0.1% LiBr at a flow rate of 1.0 ml/min.

FIG. 4 illustrates GPC chromatograms of retention time (in minutes) versus radiometric activity for radiolabelled polymers of the present invention. FIG. 4A is a chromatogram of a PG-Hex-DTPA-Gd complex according to an embodiment of the present invention labeled with ¹¹¹In. FIG. 4B is a PEG/PG-Hex-DTPA-Gd complex according to an embodiment of the present invention labeled with ¹¹¹In. The GPC was performed on a Phenomenex Biosep SEC-S300 column eluted with PBS containing 0.1% LiBr at a flow rate of 1.0 ml/min.

FIG. 5 illustrates MRIs of mice both before and two hours after intravenous injection of a PG-Hex-DTPA-Gd polymer according to an embodiment of the present invention at a dose of 0.02 mmol Gd/kg. FIG. 5A is an anterior coronal image in which “H” indicates the heart and “PV” the portal vein. FIG. 5B is a posterior coronal image in which “IVC” indicates the inferior vena cava and “K” indicates the kidney.

FIG. 6 shows signal intensity over 400 sec in the IVC (A) and in the tumor (B) following administration of PG-Hex-DTPA-Gd at a dose of 0.06 mmol/kg. Human MDA-MB-435 tumor was inoculated subcutaneously in the chest wall.

FIG. 7 illustrates a method of forming a PG-Hex-BzDTPA-Gd complex according to an embodiment of the present invention.

FIG. 8 is a GPC chromatogram of a PG-Hex-BzDTPA-Gd complex according to an embodiment of the present invention showing retention time (in minutes) versus normalized light signal intensity. The solid line represents light scattering, while the dashed line represents refractive index. The GPC was performed on a TSK-G3000PW column eluted with PBS containing 0.1% LiBr at a flow rate of 1.0 ml/min.

FIG. 9 is a GPC chromotogram of PG (Mn 17,500)(A) and PG-Hex-BzDTPA-Gd (Mn 21,950) (B) at different times after incubation of the polymer with Cathepsin B at 25° C. The elution profiles were monitored with a refractive index detector. FIG. 9A shows GPC chromatograms of PG. FIG. 9B shows GPC chromatograms of PG-Hex-BzDTPA-Gd.

FIG. 10 compares the degradation kinetics of PG (Mn 17,500), PG-Hex-BzDTPA-Gd (Mn 21,950) and PG-Hex-DTPA-Gd (Mn 222,800). The Y axis represents ratios of Refractive Index signal intensity at various time intervals to signal intensity at time zero.

FIG. 11 illustrates a method of forming a PG-BzDTPA-Gd complex according to an embodiment of the present invention.

FIG. 12 shows the percent of initial molecular weight of PG (Mn 42,000), PG-Hex-DTPA-Gd (Mn 222,800), PG-Hex-BzDTPA-Gd (Mn 21,900) and PG-BzDTPA-Gd (Mn 101,200) remaining at different times after incubation of the polymer with Cathepsin B at 25° C. Measurements were made using GPC chromatography.

FIG. 13 shows a representative spin-echo T1-weighted transverse MRI image of a tumor inoculated subcutaneously in the back of a nude mouse. The mouse was injected intravenously with PG-Bz-DTPA-Gd. Four days after contrast injection, T₁-weighted MR images (TE/TR=15/1000 ms, 4 cm field of view, 1 mm section, 256×256 matrix) were obtained on a 4.7T/40 cm MR scanner (Bruker Biospin Corp., Billerica, Mass.) with 950 mT/m, 5.7 cm inner diameter actively shielded gradient coil system (19,000 mT/m-s slew rate) and a 3.5 cm inner diameter volume radiofrequency coil for FIG. 13A. Signal enhancement in the MRI image (arrows) closely correlated to necrotic areas (arrows) in the tumor as revealed in the hematoxylin-eosin-stained tumor section in FIG. 13B.

FIG. 14 shows the percentage of necrotic areas determined by signal enhancement in tumors of mice treated with an experimental anticancer agent at different times after the initiation of drug treatment.

FIG. 15 depicts how the EMSANTT (enzyme-mediated signal amplification and neighboring-tissue trapping) effect might lead to enhanced detection sensitivity. Schematics depicting the concept of enzyme-mediated signal amplification and neighboring-tissue trapping effect. The polymeric imaging probe is initially taken up by the tumor via enhanced permeability and retention effect. Target enzymes acts on the Gd-chelated polymer upon its entering the tumor microenvironment. A large number of the enzyme-catalyzed products are transported to the necrotic region where they are trapped by a designated mechanism (A). Spatial differentiation of the enzyme-catalyzed products from the original Gd-chelated polymer allows simultaneous detection of the enzyme activity and precise delineation of the necrotic tissues with high detection sensitivity. For comparison, the effect of a hypothetical imaging probe responsive to enzymatic activity is also shown (B). Similar to the previous case, the imaging probe is initially trapped in the viable tumor area and is subsequently converted to secondary products there. Because the enzyme-catalyzed products are retained at the same site as the substrate, it is not possible to determine which of the two species are responsible for the detected MR signal.

FIG. 16 shows a T1-weighted axial image of healthy female CD1 mouse prior to injection of contrast agent into the tongue of the mouse (FIG. 16A); T2-weighted axial image of this animal following injection and repositioning (FIG. 16B); axial T1-weighted image acquired 10 minutes after injection (FIG. 16C); axial T1-weighted image acquired approximately 90 minutes after injection (FIG. 16D). Arrows indicate location of deep cervical lymph node.

### DETAILED DESCRIPTION

The present invention includes paramagnetic material and PG polymer complexes, many of which are useful in MRI as contrast agents, such as blood pool agents, and for other biological purposes. In preferred embodiments, the complex includes a paramagnetic material, such as a metal, and a PG polymer. A chelating agent may also be used to join the paramagnetic material and the PG polymer.

PG is a synthetic poly(amino acid) that is readily degraded by lysosomal enzymes to its basic component, L-glutamic acid, which is a nontoxic natural compound. The polymer has a large number of carboxyl functional grounds for attachment of other compounds. The PG polymer complex in the present invention is superior in certain ways to many polymers previously used in MRI contrast agents or for other applications in conjunction with paramagnetic materials. For example, unlike poly(lysine), which has multiple positive charges, PG has multiple negative charges, which are less biologically disruptive. PG-based MRI contrast agents also show increased safety, relaxivity, blood circulation time and other desirable qualities when compared with many other polymer-based MRI contrast agents.

PG polymers used in the present invention may vary in size. In most applications, a sample of a complex of the present invention may include a range of PG polymer sizes. However, the size or size range of the PG polymer may be selected for a particular use. For example, size influences the rate at which the polymer may move into tissues or how well the contrast agent pools in blood vessels. In an exemplary embodiment, the PG polymer has a number average molecular weight of approximately 2,000 to 100,000.

PG polymers used in the present invention may also be branched, which additionally influences their properties as contrast agents. In general branched polymers will have properties similar to longer, unbranched polymers. Branching may also be used to insert cleavage sites and thereby control the nature of degradation products as well as the rate of degradation.

The present invention also includes the use of modified PG. For instance, PEG, a nonionic highly flexible polymer, may be grafted to the PG. PEG has been shown to have numerous beneficial characteristics when used in biological systems including improved biocompatibility, increased circulation half-life, decreased immunogenicity and enhanced solubility and stability. PEG also decreases the recognition of molecules by the reticuloendothelial system and thus reduces liver uptake.

PEG may be grafted to a PG polymer by any of a number of methods to take advantage of the beneficial characteristics of PEG listed above or to obtain other benefits. The method of grafting may influence the stability of the PEG-PG complex and thus the duration of these benefits. Additionally, the method of grafting may influence the way in which the PEG-PG complex degrades. For example, in a preferred embodiment, PEG is attached to the PG side chain via an amide linkage. Because the amide linkage is relatively stable, the complex tends to degrade along the PG backbone rather than by removal of PEG from the backbone. The reactive group for attachment of metal chelators may be initially located on the PEG, the PG side chain, or both.

Any size of PEG may be attached to the PG polymer. In a preferred embodiment, each individual PEG molecule has a molecular weight of at least approximately 3,000. In a more preferred embodiment, the PEG has a molecular weight of approximately 5,000. Higher molecular weight PEG in general prolongs circulation of the modified PG polymer.

Additionally, the number of PEG molecules attached to a PG polymer may also be varied. Both number of paramagnetic materials attached and number of PEG molecules attached have positive effects on the usefulness of the PG polymer complex as a contrast agent. However, because these effects are different (paramagnetic material increases signal, while PEG increases circulation time), one effect may be sacrificed in order to increase the other effect as dictated by the particular tissue to be studied using the contrast agent.

In a preferred embodiment, the ratio of PEG to paramagnetic material or unaltered amino acid side chains is as follows: 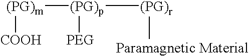
where m is at least 50, p is between 0 and 10 and r is at least 5.

In exemplary embodiments, the completed PG polymer complex, with paramagnetic material attached and any PEG attachments, has a number average molecular weight of between 10,000 and 500,000.

The paramagnetic material of the present invention may be selected based upon the intended use of the composition. In another exemplary embodiment of the present invention, the paramagnetic material is suitable to increase contrast during an MRI. For example, the paramagnetic material may be Gd, Mn or iron oxide.

The paramagnetic material may be coupled to the PG polymer through any possible covalent linkage. In an exemplary embodiment, the paramagnetic material is bound to a chelating agent, which is covalently bonded with the PG polymer. The chelating agent may be selected based upon the paramagnetic material to be bound, ease of covalent bonding with the PG polymer, and toxicity of the chelating agent or its breakdown products. In embodiments where the paramagnetic material is Gd, DTPA is a suitable chelating agent. The chelating agent may be directly bound to the carboxyl chain of the PG or it may be linked using another molecule, such as a alkyl linker or S—S disulfide linker. In an exemplary embodiment, the alkyl linker is between 2 and 12 units in length, such as a 6-aminohexyl group. Short PEG molecules containing, for example only approximately 1-12 monomers may also be used. Use of a linking molecule can influence the way in which the overall molecule is degraded.

In exemplary embodiments of the present invention, DTPA is used as a chelating agent. However, dianhydride forms of DTPA can bond with two separate PG monomers and thereby crosslink two separate PG polymers. This crosslinking results in a larger overall polymer structure. (See Table 1.) Although in certain applications high molecular weight polymers (>100,000 D) are preferable, in other cases it is desirable to be able to eliminate or substantially reduce polymer crosslinking and to control the polymer molecular weight.

Chelators which are often used to bind metal ions include but no limited to diethylenetriaminepentaacetic acid (DTPA), p-aminobenzyl-diethylenetriaminepentaacetic acid (p-NH₂-Bz-DTPA), ethylene diaminetetracetic acid (EDTA), 1,4,7,10-tetraazacyclododecane-N,N′,N″,N′″-tetraacetic acid (DOTA), 2-p-aminobenzyl-1,4,7,10-tetraazacyclododecane-N,N′,N″,N′″-tetraacetic acid (p-NH₂-Bz-DOTA), 1,4,7,10-tetraazacyclododecane-1,4,7,10-tetrakis(methylene phosphonic acid) (DOPA), and 3,6,9,15-tetraazabicyclo[9.3.1]pentadeca-1(15),11,13-triene-3,6,9-triacetic acid (PCTA).

Embodiments of the present invention may also include targeting molecules, such as homing ligands, to further direct biological location of the PG polymer complex. These molecules may be attached to side chains of the PG backbone or to modifications of the backbone, such as PEG molecules attached as described above. In a specific embodiment, the homing ligands may target neovasculature. Homing ligands may include agents that home to growth factor receptors, cytokines and integrins, inter alia.

Complexes of the present invention employing only a PG polymer without PEG modification may be synthesized by first providing a solution of PG of the desired size or size range. The carboxyl groups of the PG side chains may then be modified, if necessary, before bonding with the chelating agent. The percentage and total number of carboxyl groups altered in the manner may change the properties of the ultimate composition. In an exemplary embodiment, at least approximately 50% of the carboxyl groups are modified.

Next, the chelating agent is provided under conditions allowing covalent bonding of the chelating agent to the modified side chain. The percentage or number of side chains to which the chelating agent is attached influences the ultimate properties of the composition. In a preferred embodiment of the composition, at least 50% of the modified side chains bond to the chelating agent.

Finally, the paramagnetic material to be chelated is added to the mixture.

A PEG-grafted PG complex may be formed in a similar manner except that PEG may be added to some PG side chains prior to addition of the chelating agent. Complexes of the present invention may be used as MRI contrast agents to image any suitable tissue. However, the agents are particularly well suited for imaging that benefits from a blood pool contrast agent. For example, complexes of the present invention may be used to detect anatomy, altered anatomy, vasculature leakages or occlusion, ulcers, angiogenesis, such as that in metastatic tumors, neoplasms, infection and inflammation. The complexes may also be used to detect vascular occlusion such as that caused by thrombosis or atherosclerotic lesions. Absent a contrast agent, these features are difficult to detect with MRI or may not be detected in sufficient detail to allow better diagnosis and development treatment protocol.

In a specific embodiment, the present invention may be used to image therapy-induced necrosis of a tumor. More particularly, PG-Bz-DTPA-Gd may be used for this purpose.

In another specific embodiment, the present invention may be used for mapping of lymph node drainage and identification of lymph node metastases. More specifically, PG-DTPA-Gd may be used for such mapping.

The following examples are provided to further describe selected embodiments of the present invention.

### EXAMPLES

### Example 1

### Materials and Analytical Methods

In these examples, materials were obtained from the following sources: PG sodium salt; N-tert-butoxycarbonyl-1,6-diaminohexane hydrochloride (t-Boc-Hex-NH₂); 1,3-diisopropylcarbodiimide; pyridine; 4-dimethylaminopyridine; trifluoroacetic acid (TFA); diethylenetriaminepentaacedic acid (DTPA) dianhydride; gadolinium (III) chloride hexahydrate; 2,4,6-trinitrobenzenesulfonate (TBNS); 4-(2-pyridylazo)resorcinol (PAR); PBS (0.01 M phosphate buffered saline containing 138 mM NaCl and 2.7 mM KCl, pH 7.4) Cathepsin B and all other reagents not otherwise indicated in this example and solvents were purchased from Sigma Aldrich (St. Louis, Mo.).

Succinimidyl ester of methoxy poly(ethylene glycol)propionic acid (mPEG-NHS) was purchased from Shearwater (Huntsville, Ala.).

¹¹¹InCl₃ was obtained from Perkin-Elmer Life Sciences (Boston, Mass.).

p-Isothiocyanatobenzyl-diethylenetriaminepentaacetic acid (p-SCN-Bz-DTPA′3HCl) and p-aminobenzyl-diethylenetriaminepenta(aceticacid-t-butyl ester) [NH₂-BzDTPA-penta(t-butyl)]were obtained from Macrocyclics (Dallas, Tex.).

Spectra/Pro 7 dialysis tubing with molecular weight cutoff (MWCO) of 10,000 and Centricon Plus-20 centrifugal filters were purchased from Fisher Scientific (Pittsburgh, Pa.).

PD-10 columns were purchased from Amersham-Pharmacia Biotech (Piscataway, N.J.).

Materials with similar properties obtained from other commercial or non-commercial sources may also be used in the present invention.

All gel permeation chromatography (GPC) in these examples was performed on a Waters (Milford, Mass.) high-performance liquid chromatography (HPLC) system including a 600 controller, as 717 plus auto sampler, and a Viscotek E-Z^{pro} triple detector (Viscotek, Houston, Tex.) that records refractive index, viscosity, and light-scattering signals. Samples were separated using a TSK-G3000PW column (TosoHaas, Montgomeryville, Pa.) eluted with PBS containing 0.1% LiBr at a flow rate of 1.0 ml/min. Number-average molecular weights of the polymer conjugates were calculated using Viscotek TriSEC GPC software. Radio-GPC was performed with a HPLC unit equipped with a LDC pump (Laboratory Data Control, Rivera Beach, Fla.) and a LUDLUM radiometric detector (Measurement Inc. Sweetwater, Tex.). The samples were separated using a Phenomenex Biosep SEC-S3000 7.8 mm×30 cm column eluted with PBS containing 0.1% LiBr at a flow rate of 1.0 ml/min. ¹H NMR was recorded at 300 MHz on a Bruker Avance 300 spectrometer (Billerica, Mass.) using D₂) as a solvent. Elemental analysis was performed by Galbraith Laboratories, Inc. (Knoxville, Tenn.). Free amino groups in the polymer conjugates were quantified using a TNBS assay following a reported protocol (Hermanson G T, Bioconjugate Techniques, San Diego, Calif.: Academic Press (1996) pp. 112-3.)

Similar GPC equipment and methodologies may also be used in the present invention.

### Example 2

### Synthesis of 6-aminohexyl Poly(L-glutamine)

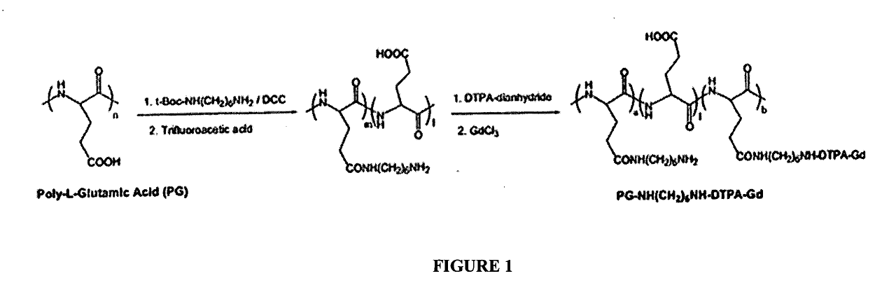

As part of a method of the present invention, PG functionalized with one or more 6-aminohexyl groups was prepared. The resulting molecule was of the general formula PG[-NH(CH₂)₆NH₂]ₙ, where n is greater than or equal to one. As shown in FIG. 1, the 6-aminohexyl group is attached to a side chain of a glutamic acid residue via the carboxyl group.

To prepare the 6-aminohexyl functionalized PG first an aqueous solution of sodium salt of PG with a number average molecular weight (Mₙ) of 17,000 and a degree of polymerization of 116 was converted to its acid form by acidifying with 1 N HCl to pH 3-4. The polymer precipitate was collected by centrifugation, washed with deionized water and lyophilized. 1.3 g of the lyophilized polymer was next added to 20 ml of anhydrous dimethylformamide to prepare a 10 mmol [COOH] solution. To this solution was added 2.0 g (8 mmol) of t-Boc-Hex-HN₂ hydrochloride, 1.0 g (8 mmol) 1,3-diisopropylcarbodiimide, 0.1 ml of pyridine and a trace amount of 4-dimethylaminopyridine. The reaction mixture was stirred at room temperature overnight, then the precipitate was filtered and remaining solvent removed under vacuum. The residue was dissolved in 20 ml trifluoroacetic acid in an ice bath and stirred at 4° C. overnight. Excess trifluoracetic acid was removed under vacuum. After dissolving the residual solid in ice water followed by neutralization with 1 N NaOH, the solution was dialyzed against PBS and deionized water using a MWCO of 10,000. The dialysate was lyophilized to yield 1.5 g (72%) of a sponge-like solid. About 46 of the 116 glutamic acid residues in the PG, or about 40%, were converted to 6-aminohexyl glutamine. Calculations of conversion percentages were based on the ratio of the integrals of 6-CH₂ protons adjacent to the amine in the 6-aminohexyl group (δ=2.88 ppm, t) to the α-CH proton in the PG backbone (δ=4.17 ppm, s).

### Example 3

### Synthesis of PG-Hex-DTPA-Gd

Also as shown in FIG. 1, the DTPA of PG-Hex-DTPA-Gd is conjugated to the 6-aminohexyl group of the PG side chains. To prepare a PG-Hex-DTPA-Gd complex, an approximately 1.1 mmol [NH₂] solution of 6-aminohexyl PG prepared as described above was prepared by dissolving 500 mg of the compound in 15 ml of 0.1 M NaHCO₃. To this solution, 1.43 g of DTPA-dianhydride (4 mmol) was added in portions over a period of 30 minutes. The pH of the reaction solution was adjusted to 8 by adding aliquots of 0.1 N NaOH solution over the 30 min period. After stirring at room temperature for two hours, the reaction mixture was dialyzed against PBS and deionized water at MWCO 10,000. The resulting solution was concentrated to 5 ml on a centrifugal filter at MWCO 10,000 and stored at 4° C. for future use. The amount of DTPA attached to the PG was determined by quantifying unreacted amino groups with a TNBS assay. Approximately 60% of the amino groups available at the start of the reaction (those at the end of the 6-aminohexyl groups) were conjugated to DTPA. Accordingly, because approximately 46 6-aminohexyl groups per PG were available initially, this resulted in a degree of DTPA substitution on the PG polymer of about 0.24 mol DTPA/mol of COONa initially available on the PG. See Table 1.

To chelate PG-Hex-DTPA with Gd³⁺, a 100 mg/ml solution of GdCl₃.9H₂O in water was added drop-wise to an aqueous solution of PG-Hex-DTPA at approximately 50 mg/ml (10 ml). The presence of unchelated Gd³⁺ in the mixture was monitored with a Gd³⁺ indicator, 4-(2-pyridylazo)resorcinol. The yellowish color of resorcinol solution turns red in the presence of trace amounts of unchelated Gd³⁺ ion. Approximately 2.2 ml of GdCl₃ (0.78 mmol) was needed to completely convert the DTPA in the sample to DTPA-Gd. The reaction solution was dialyzed against water at MWCO 10,000 until no free Gd was detected in the receiving medium. The product was further purified by gel filtration on a PD-10 column. Fractions that showed only a single polymer peak on GPC were pooled and lyophilized to result in 1.0 g of product 92%. The compound contained 11.9% (w/w) of Gd as determined by elemental analysis.

Various physiochemical properties of the PG-Hex-DTPA-Gd are summarized in Table 1. As the data in Table 1 indicate, the measured molecular weight of the PG-Hex-DTPA-Gd was much greater than that calculated theoretically. The polydispersity of the chelated polymer (M_{w}/Mₙ=4.7) was greater than that of the unsubstituted polymer (M_{w}/Mₙ=1.2). The relaxivities, expressed as R₁ and R₂ for the Gd³⁺-chelated polymers determined at 1.5 Tesla were 3-5 fold greater than those of DTPA-Gd.

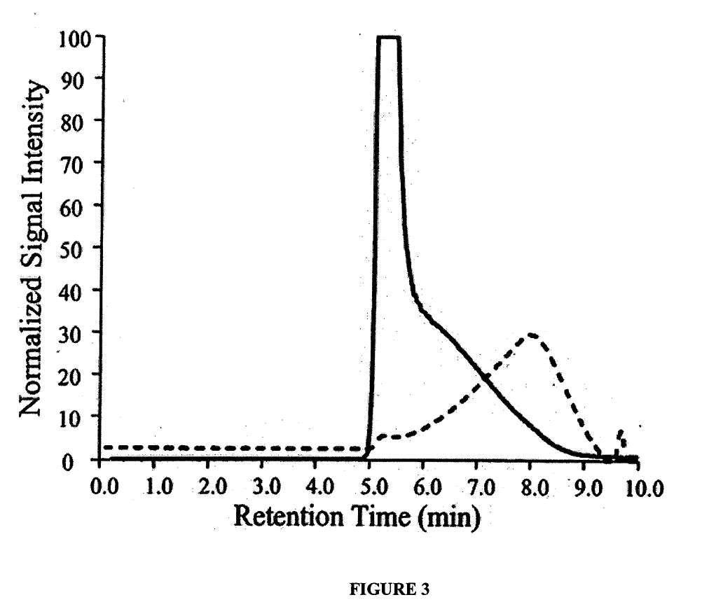

Furthermore, as shown in FIG. 3, PG-Hex-DTPA-Gd exhibits as strong peak in its light scattering profile, but not in its refractive index profile or its viscosity profile (not shown in the figure).

During conjugation of DTPA-anhydride with 6-aminohexyl PG, some crosslinking can take place. This may explain the high measured molecular weight of PG-Hex-DTPA-Gd as compared to the calculated theoretical molecular weight. This is also consistent with the GPC profile of the complex, such as that shown in FIG. 3. PG-Hex-DTPA-Gd showed a strong peak at the dead volume of the column when monitored with a light scattering detector, indicating the presence of a fraction of ultra-high molecular weight, cross-linked polymer. However, the GPC profile obtained with a refractive index detector revealed only a small spike at the retention volume corresponding to the strong light scattering signal. This indicates that the cross-linked polymer represents only a small fraction of the polymeric mass. Cross-linking is likely minimized in the present example because DTPA was added to the 6-aminohexyl PG slowly.

### Example 4

### Synthesis of PEG/PG

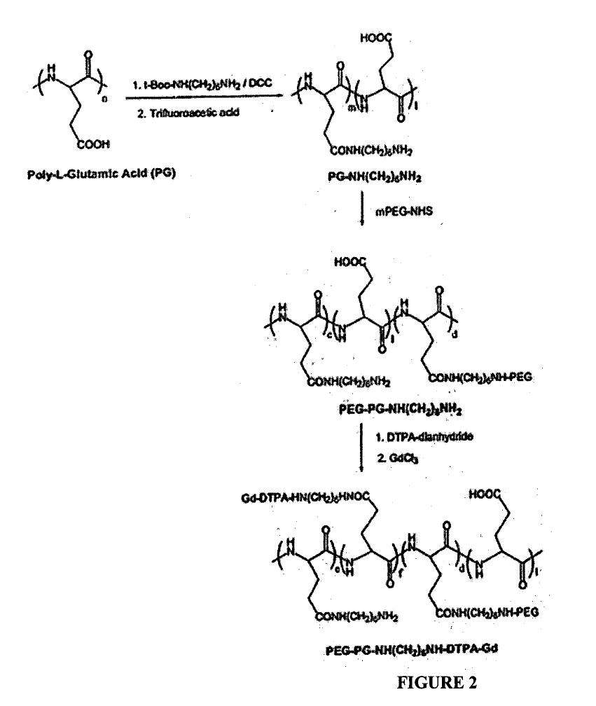

As shown in FIG. 2 after addition of 6-aminohexyl to PG, but before addition of DTPA-dianhydride, PEG may be grafted onto the PG through the 6-aminohexyl groups. To prepare PEG/PG, an approximately 0.66 mmol [NH₂] solution of 6-aminohexyl PG (300 mg) in 0.1 N Na₂HPO₄ (10 mL) at pH 8.0 was prepared. 300 mg (0.06 mmol) mPEG-NHS (MW 5,000) was added to the solution in small portions. The reaction mixture was stirred at room temperature overnight. Unreacted PEG was removed by ion-exchange chromatography using an AKTA fast-protein liquid chromatography system (Amersham-Pharmacia Biotech) equipped with a Hiload Sepharose Q16/10 HP column (Pharmacia). The samples were eluted with 20 mM Tris buffer (pH 8.0) and a linear gradient of 0%-100% 2 N NaCl in 15-column volume at a flow rate of 3 ml/min. The product was further purified by dialysis against deionized water at MWCO 10,000. Lyophilization of the dialysate yielded 240 mg of product (40%). ¹H NMR analysis indicated that approximately four PEG molecules were conjugated to each PG polymer chain. This is approximately 0.034 mol PEG/mol COONa initially available on the PG.

### Example 5

### Synthesis of PEG/PG-Hex-DTPA-Gd

PEG/PG-Hex-DTPA-Gd was prepared using the procedures used for preparation of PG-Hex-DTPA-Gd in Example 3, but with PEG/PG as the starting material, as shown in FIG. 2. Specifically, and approximately 0.12 mmol [NH₂] solution of PEG/PG (120 mg) was reacted with 0.4 mmol DTPA-dianhydride (140 mg) and then chelated with GdCl₃ to produce PEG/PG-DTPA-Gs with an overall yield of 33%. Fifty percent of the amino groups available on the PG with 6-aminohexyl were conjugated to DTPA as determined by colorimetric assay. Accordingly, approximately 23 DTPA chelators were attached to each PG. This is 0.20 mol DTPA/mol COONa initially available on the PG. This lower amount of DTPA attached to the PG as compared to the complex lacking PEG is likely due to steric hindrance by the grafted PEG molecules. The conjugate contained 7.26% Gd (w/w) as determined by elemental analysis.

Physiochemical properties of the Gd³⁺-chelated PEG/PG are summarized in Table 1. PEG/PG-Hex-DTPA-Gd has higher relaxivities than did PG-Hex-DTPA-Gd. PEG/PG-Hex-DTPA-Gd exhibited a GPC chromatograph profile very similar to that of PG-Hex-DTPA-Gd shown in FIG. 3. GPC chromatographs for PEG/PG-Hex-DTPA-Gd also revealed a small amount of cross-linked polymer.

TABLE 1
PHYSICOCHEMICAL PROPERTIES OF PG-HEX-DTPA-GD
AND PEG-HEX-DTPA-GD

| | PG-Hex- | PEG/PG-Hex- | |
|---|---|---|---|
| Property | DTPA-Gd | DTPA-Gd | DTPA-Gd |
| Number-average | | | |
| MW | | | |
| Calculated | 36,400 | 53,700 | 560 |
| Measured | 222,800 | NA | |
| Polydisp. | 4.70 | NA | |
| Degree of | | | |
| substitution | | | |
| (mol/mol of | | | |
| COONa) | | | |
| NH₂ | 0.40 | 0.40 | |
| PEG | | 0.034 | |
| DTPA | 0.24 | 0.20 | |
| Gd content, % | 11.9 | 7.26 | 28.5 |
| (w/w) | | | |
| Relaxivity | | | |
| R₁ (mM⁻¹Sec⁻¹) | 12 | 20.5 | 4.1 |
| R₂ (mM⁻¹Sec⁻¹) | 18 | 27.3 | 5.2 |

Calculated number-average molecular weight (MW) was calculated on the basis of degree of substitution. For PG alone, the number-average molecular weight was 17,500 and the degree of polymerization was 116.

Polydispersity was calculated as the measured molecular weight divided by the number-average molecular weight.

Degree of substitution with NH₂ and PEG was measured by ¹H nuclear magnetic resonance. Degree of substitution of DTPA was measured by quantifying the concentration of NH₂.

Gd content was measured by elemental analysis.

Relaxivity was measured at 1.5 Tesla. Relaxivity date for DTPA-Gd was obtained from Unger, E. C., et al. Magnetic Resonance Materials in Physics, Biology and Medicine, 8: 154-162 (1999), where relaxation times were determined at 1.5 Tesla in phosphate-buffered saline.

### Example 6

### Determination of Relaxivity

Solutions of either PG-Hex-DTPA-Gd of PEG/PG-Hex-DTPA-Gd were prepared in saline at Gd concentration of 0.005, 0.01, 0.02, 0.04 and 0.08 and 0.16 mM. Spin lattice (T₁) and spin-spin (T₂) relaxation times were measured at 1.5 Tesla on a GE Sigma LX scanner (Milwaukee, Wis.) using inversion recovery and multiecho T₂-weight pulse sequences. Relaxivities (R₁ or R₂ in mM₋₁s₋₁ were obtained from linear least square determination of the slopes of 1/T₁ versus [Gd] or 1/T₂ versus [Gd] plots.

### Example 7

### ¹¹¹In-Labeling of PG-GD Complexes

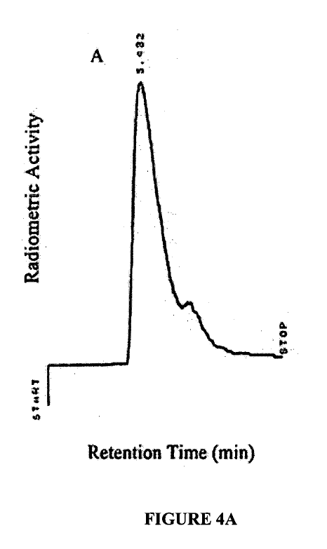

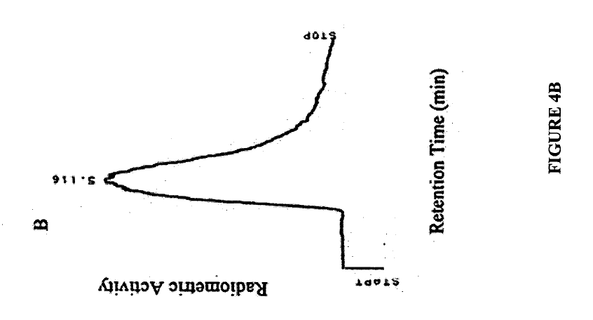

An aqueous solution of either PG-Hex-DTPA-Gd (100 mg/ml, 0.1 ml) or PEG/PG-Hex-DTPA-Gd (44 mg/ml, 0.3 ml) in 1 N sodium acetate buffer (pH 5.5) was incubated with 300 μCi of ¹¹¹InCl₃ at room temperature for 15 minutes. Free ¹¹¹In was removed by gel filtration on a PD-10 column eluted with PBS. The purity of the radiolabelled compounds was analyzed by radio-GPC. Both Gd-PG complexes were successfully radiolabelled as demonstrated by the absence of small-molecular-weight ¹¹¹In-DTPA (retention time 9.34 min) in the CPC chromatograms of the radiolabelled polymers presented in FIG. 4.

### Example 8

### Biodistribution Studies

Female C3Hf/Kam mice (20-25 g) were bred and maintained in a specific pathogen-free mouse colony at the Department of Experimental Radiation Oncology at the University of Texas M.D. Anderson Cancer Center. All experiments involving animals were performed in accordance with the guidelines of the Institutional Animal Care and Use Committee. Solid murine MCa-4 tumors were generated in the muscles of the right thigh of mice by inoculating each with 5×10⁵ tumor cells in suspension in PBS. When tumors has grown to 8 mm in average diameter, mice were randomly allocated into groups, each group consisting of 4 mice. Mice in each group were injected intravenously with either ¹¹¹In-labeled PG-Hex-DTPA-Gd (0.5 mg, 2 μCi per mouse) or ¹¹¹In-labeled PEG/PG-Hex-DTPA-Gd (0.5 mg, 5 μCi per mouse). Animals were killed at 5 minutes, 2 hours, or 24 hours after injection. Blood, liver, spleen, kidney, muscle and tumor tissues were removed, weighed, and counted for radioactivity with a gamma counter (Packard, Ill.). Uptakes of contrast agent in various tissues were calculated as the percentage of the injected dose per gram of tissue. Student's t test was used to compare differences in tissue uptakes between the two agents, with p values less than 0.05 considered significant.

Biodistribution data for radiolabelled PG-Gd complexes are summarized in Table 2. Both contrast agents were slowly cleared from the blood. At 2 hours after intravenous injection, the uptake in the blood (expressed as the percentage of the injected dose by radioactivity per gram of blood) was 16% for radiolabelled PG-Hex-DTPA-Gd and 10% for radiolabelled PEG/PG-Hex-DTPA-Gd. The highest uptake of both contrast agents was found in the kidney. Between 2 hours and 24 hours after injection, activities of radiolabelled PG-Hex-DTPA-Gd in the liver, spleen and kidney dropped 3.7%, 18.8% and 16.6%, respectively. In contrast, activities of radiolabelled PEG/PG-Hex-DTPA-Gd in the liver and spleen increased over the same period. Additionally, a marked increase of uptake of both radiolabelled complexes in tumors was observed between 5 minutes and 24 hours after injection. Specifically, the increase for PG-Hex-DTPA-Gd was 217%, while the increase for PEG/PG-Hex-DTPA-Gd was 41%. The difference in increased tumor uptake between the two polymer complexes may be a result of longer circulation time and enhanced permeability and retention of PG-Hex-DTPA-Gd in the tumor as compared to PEG/PG-Hex-DTPA-Gd. At two hours after injection radiolabelled PG-Hex-DTPA-Gd has significantly less activity in the liver (p<0.001) and more activity in the blood (p<0.001), kidney (p=0.008) and tumor (p=0.006) than did radiolabelled PEG/PG-Hex-DTPA-Gd.

TABLE 2
BIODISTRIBUTION OF GD³⁺-CHELATED PG POLYMERS

| | | | | | | |
|---|---|---|---|---|---|---|
| Time | Blood | Liver | Spleen | Kidney | Muscle | Tumor |

PG-DTPA-Gd

| | | | | | | |
|---|---|---|---|---|---|---|
| 5 min | 26.86 +/− 4.15 | 4.28 +/− 0.94 | 6.35 +/− 1.70 | 15.32 +/− 4.85 | 0.39 +/− 0.04 | 1.07 +/− 0.38 |
| 2 hours | 16.25 +/− 1.68 | 7.48 +/− 0.38 | 9.32 +/− 1.14 | 52.69 +/− 7.35 | 0.42 +/− 0.07 | 2.93 +/− 0.34 |
| 24 hours | 4.78 +/− 1.24 | 7.20 +/− 1.48 | 7.57 +/− 1.65 | 43.83 +/− 4.28 | 0.44 +/− 0.08 | 3.39 +/− 1.10 |

PEG/PG-DTPA-Gd

| | | | | | | |
|---|---|---|---|---|---|---|
| 5 min | 42.61 +/− 3.83 | 7.78 +/− 1.16 | 4.87 +/− 0.39 | 14.69 +/− 0.98 | 0.57 +/− 0.12 | 1.62 +/− 0.76 |
| 2 hours | 10.17 +/− 0.78 | 19.80 +/− 1.28 | 10.56 +/− 0.66 | 37.34 +/− 2.77 | 0.51 +/− 0.05 | 1.95 +/− 0.32 |
| 24 hours | 1.43 +/− 0.13 | 21.10 +/− 0.79 | 12.36 +/− 1.18 | 36.41 +/− 3.10 | 0.34 +/− 0.04 | 2.28 +/− 0.30 |

All data other than time are presented as percentage of injected dose per gram of tissue. Each value represents mean+/−standard deviation (n=4). “Time” indicated time after intravenous injection of polymer.

### Example 9

### MRI Studies

For MRI studies using complexes prepared as described in the earlier examples, mice were anesthetized with 1-2% isoflurane in a 1 L/min O₂ flow and place prone within a custom sled. A custom catheter made from 2 Fr silicon tubing and a 27 gauge needle was inserted in the tail vein of the mouse. Gd-DTPA (0.1 mm Gd/kg) or PG-Hex-DTPA-Gd (0.004 mmol Gd/ml, 0.02 mmol Gd/kg) was injected through the catheter and the catheter flushed with normal saline. Two-dimensional coronal section spin-echo images were obtained before and at 2 hours and 24 hours after contrast agent injection using a 2.5 cm quadrature birdcage coil on a 1.5 T Sigma LX MR scanner (GE Medical Systems, WI). Six 1.5 mm contiguous sections were obtained using a 6 cm field-of-view, a 256×192 acquisition matrix and 4 averages. Similar studies were performed with PEG/PG-Hex-DTPA-Gd.

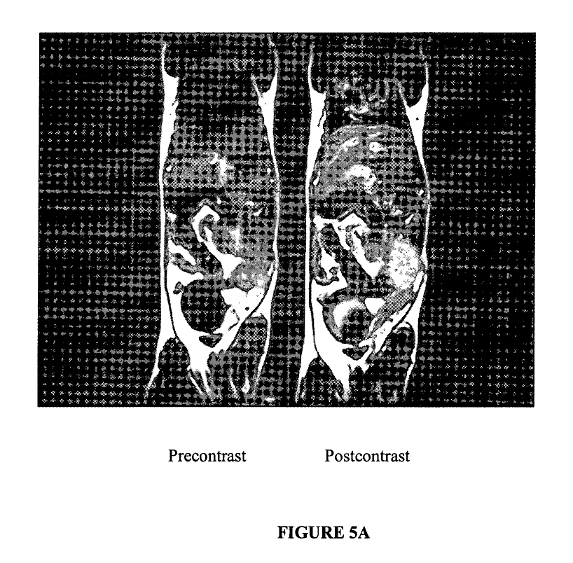

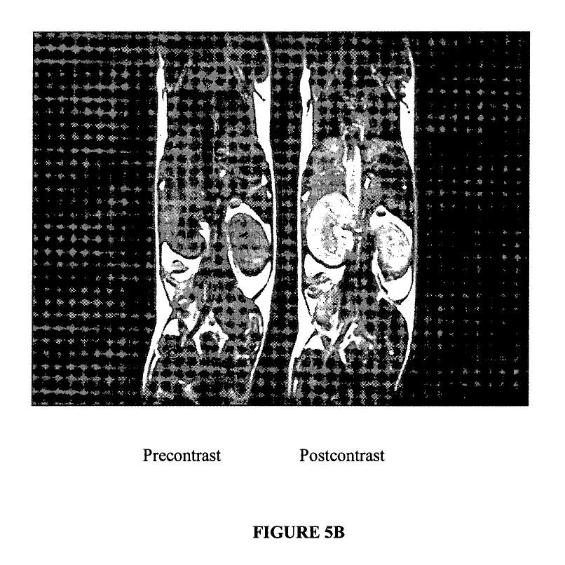

These studies showed markedly greater vascular enhancement with both of the complexes than with Gd-DTPA. Vascular enhancement was maintained two hours after injection of the complexes at a dose of 0.02 mmol Gd/kg. Blood vessels, such as the vena cava, were clearly visualized. Myocardial, hepatic and renal perfusion were also demonstrated. An example of MRI images obtained using the PG-Hex-DTPA-Gd complex are shown in FIG. 5. In FIG. 5A myocardial and hepatic perfusion can be seen. FIG. 5B shows pulmonary, hepatic and renal perfusion as well as persistent enhancement of the vena cava. In contrast, vascular enhancement was not detected 2 hours after injection of Gd-DTPA at a dose of 0.1 mmol/kg.

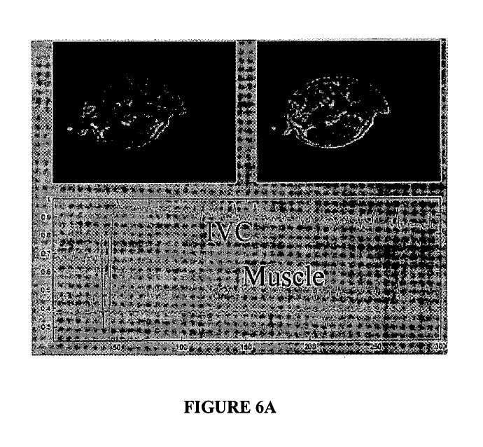

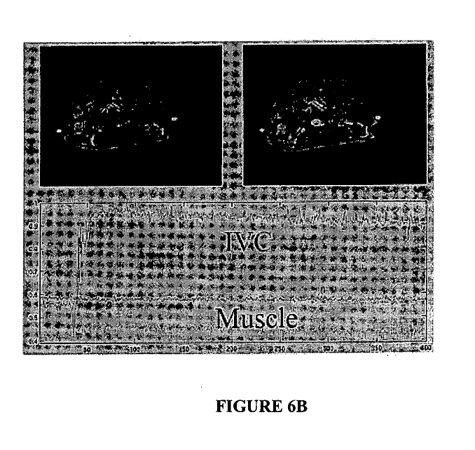

To demonstrate the long intravascular retention time for the PG-Hex-DTPA-Gd, dynamic contrast enhanced (DCE) MR images were obtained before, during, and following administration of 0.06 mmol/kg of PG-Gd-DTPA via tail vein infusion. A fast spoiled gradient-echo sequence was used to obtain the data from 8 anatomic sections with 7.1 s temporal resolution. Other acquisition parameters were echo time: 1.4 ms, repetition time: 40 ms, flip angle: 35°, acquisition matrix size: 128×128. FIG. 6A demonstrates the minimal loss of signal intensity, over 400 sec, in the IVC following administration of PG-Hex-DTPA-Gd. Such long intravascular retention times are useful for contrast-enhanced MR angiography, for assessment of microvascular volume, and for characterizing changes in permeability of highly “leaky” vasculature. Retention of the contrast in the tumor was also noted in the same time period (FIG. 6B).

### Example 10

### Acute Toxicity

A preliminary study of acute toxicity of the PG-Gd complexes of these examples were performed using two healthy Swiss mice per group. Mice received six doses of PG-Hex-DTPA-Gd with doses escalated in 2-fold increments, starting at 0.01 mmol Gd/kg and eventually reaching 0.32 mmol Gd/kg. The effects of PG-Hex-DTPA-Gd were determined by observing the number of deaths that occurred within two weeks following the contrast injection. No mouse died during the experimental period at doses of up to 0.32 mmol Gd/kg.

Because of the high loading of Gd in the polymer complexes of these examples, the doses of polymer carrier that would be needed to be introduced into the body to obtain a dose of 0.02 mmol Gd/kg are estimated to be less than 26 mg/kg for PG-Hex-DTPA-Gd and less than 43 mg/kg for PEG/PG-Hex-DTPA-Gd. These doses are 20 to 30-fold less than the dose of PG given to rodents in this example without causing apparent toxic effect.

### Example 11

### Synthesis of PG-Hex-BzDTPA-GD

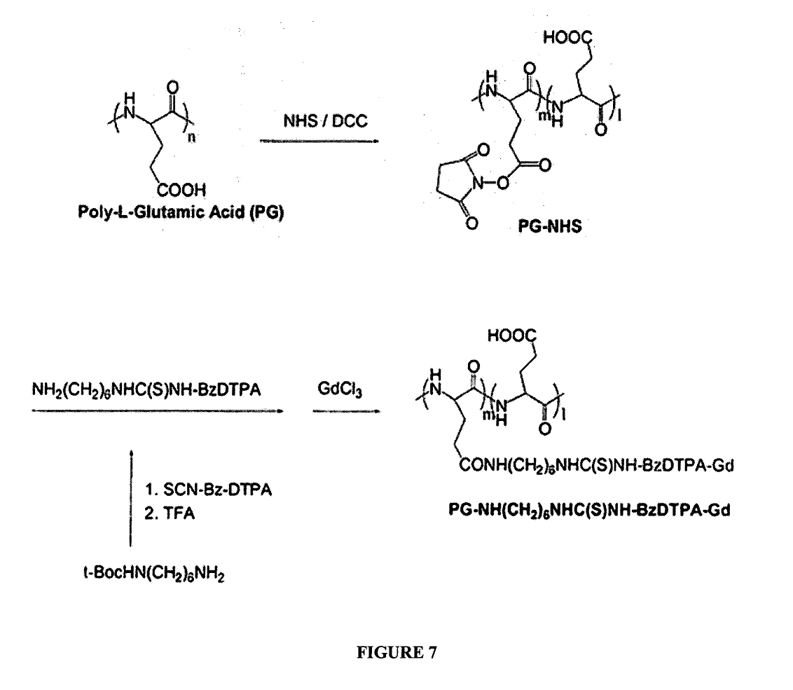

Synthesis of PG-Hex-BzDTPA-Gd is shown in FIG. 7. 0.92 mmol of t-Butyl N-(6-aminohexyl)carbamate hydrochloride was reacted with 0.46 mmol of p-SCN-Bz-DTPA′3HCl in DMF in the presence of triethylamine (0.5 ml) and stirred at 4° C. overnight. The precipitate was removed by filtration and residual solvent in the precipitate was removed under vacuum. The residue was then dissolved in chloroform and extracted with 1 M NaOH. This basic aqueous solution was acidified to pH 2-3 with 1 M HCl. The resulting precipitate was collected, washed with water (pH 2-3) and dried. Yield was 72%. Mass spectrometry results were as follows: 756.02 (calculated 756.87, C₃₃H₅₂N₆O₁₂S). ¹H-NMR (DMSO): δ 7.37 ppm (d, J=7.8, 2H, φ-H), δ 7.15 ppm (d, J=7.8, 2H, φ-H), δ 1.37 ppm (s, 9H, —CH₃), δ 1.27-1.50 ppm (m, 8H, —CH₂CH₂CH₂CH₂— in hexamethylenediamine).

This t-Boc protected product was dissolved in 2 ml of trifluoroacetic acid (TFA) and stirred at room temperature for three hours. 1 mL of water was added to quench the reaction. TFA and water was removed under vacuum. The residue was dissolved in 1 ml of methanol and precipitated with ether. The product was collected and washed thoroughly with ether and dried to give a yellowish powder (yield 92%). The product was used for the subsequent reaction without further purification.

0.79 mmol of poly-L-glutamic acid (number-average MW: 8,850, 102 mg) was reacted with 0.36 mmol of N-hydroxysuccinimide in DMF in the presence of 0.4 mmol DCC. The mixture was stirred at 4° C. overnight. The precipitate (DCU) was removed by filtration and the solution was concentrated under vacuum. Chloroform was added to precipitate the polymer. The product was collected by centrifugation, washed with chloroform, and dried under vacuum. A solution of the activated ester PG-NHS (60 mg) in DMF was added in portions into a solution of NH₂(CH₂)₆NH(CS)NH-BzDTPA (0.09 mmol) in 0.1 M NaHCO₃ (pH 8.5) over a period of 3-4 hours. The pH of the solution was maintained at 8-9 by addition of 1 M NaOH. After stirring at 4° C. overnight, DMF was removed and the aqueous solution was dialyzed against PBS and water (molecular weight cut-off, 10,000). Lyophilization of the dialysate yielded a sponge powder.

Complex with Gd was achieved following similar procedures as described in previous examples. Briefly, 65 mg of PG-NH(CH₂)₆NH(CS)NH-BzDTPA was mixed with 30 eq of GdCl₃ in 0.3 M sodium acetate and the solution was extensively dialyzed against PBS followed by dialysis against water. Yield was 65%. The polymer contained 10.6% of gadolinium (w/w). About 13 DTPA-benzylhexamethylenediamino groups were attached to each PG chain.

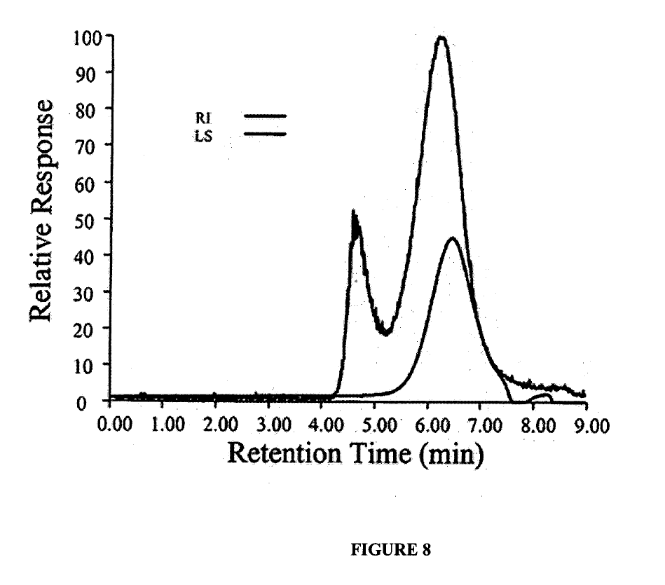

The number-average molecular weight of the resulting polymeric conjugate was 21,950 as determined by gel permeation chromatography using Viscotek E-Z^{pro} triple detector and TriSEC GPC triple detector software (Viscotek, Houston, Tex.). The molecular weight distribution of PG-Hex-BzDTPA-Gd was narrow (polydispersity: 1.32). In contrast, the Gd-chelated PG obtained using the previous method, PG-Hex-DTPA-Gd, yielded polymer with broad molecular weight distribution (polydispersity, 4.70). FIG. 8 shows a GPC chromatograms of PG-Hex-BzDTPA-Gd. When compared to FIG. 3, in which the light scattering profile of PG-Hex-DTPA-Gd shows a large peak at the dead volume of the column indicating extensive crosslinking of the polymer, that of PG-Hex-BzDTPA-Gd shown in FIG. 8 only shows a relatively small peak at the dead volume of the column. These data indicate that the synthetic method of the present example yields Gd-chelated PG polymer with much less crosslinking. The physicochemical properties of PG-Hex-BzDTPA-Gd are summarized in Table 3.

TABLE 3
PHYSICOCHEMICAL PROPERTIES
OF PG-HEX-BZDTPA-GD

| Property | PG-Hex-BZDTPA-Gd |
|---|---|
| Number-average MW | |
| Calculated | 19,420 |
| Measured | 21,950 |
| Polydispersity | 1.32 |
| DTPA | 0.45 |
| Gd content, % (w/w) | 10.6 |
| Relaxivity | |
| R₁ (mM⁻¹Sec⁻¹) | 21.8 |

Calculated number-average molecular weight was based upon degree of DTPA substitution and starting PG number-average molecular weight. A number average molecular weight of 8,850 was used for PG (degree of polymerization was 59).

All other measurements and calculations were made in the same manner as in Table 1.

PEG/PG-BzDTPA may be prepared in an analogous fashion by synthesizing NH₂(CH₂)₆NHC(S)NH-PEG, then adding this to hydroxysuccinimide-modified PG polymer prior to addition of BzDTPA.

### Example 12

### Biodegredation of PG-Hex-BzDTPA-Gd

PG-Hex-BzDTPA-Gd was dissolved in PBS buffer (pH 5) at a concentration of 5.3 mg/ml. Cathepsin B, which is able to degrade the complex, was added to the solution to a final concentration of 10 units/ml. The degradation was monitored at different time intervals using a gel permeation chromatography (GPC) system equipped with an TSK G3000PW column (TosoHaas), a Waters 515 HPLC pump, and a Viscotek E-Z^{pro} triple detector array. The system was eluted with PBS buffer containing 0.1% LiBr (pH 7.4) at a flow rate of 1 ml/min. The PG-Hex-BzDTPA-Gd was degraded when incubated with cathepsin B at RT. The degradation reached maximum at 2 hr.

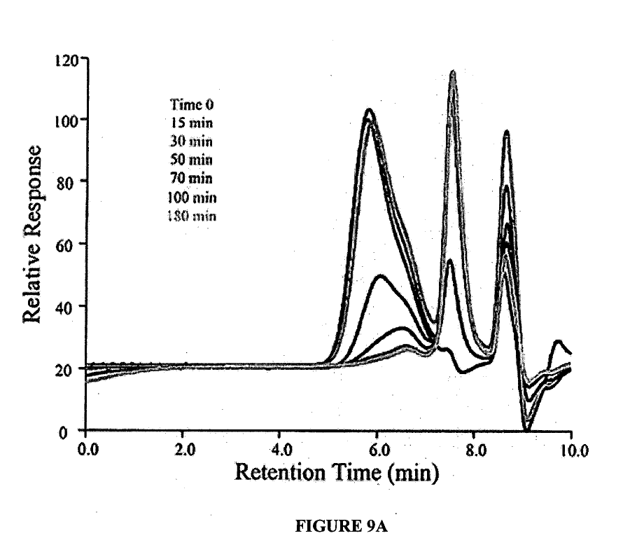

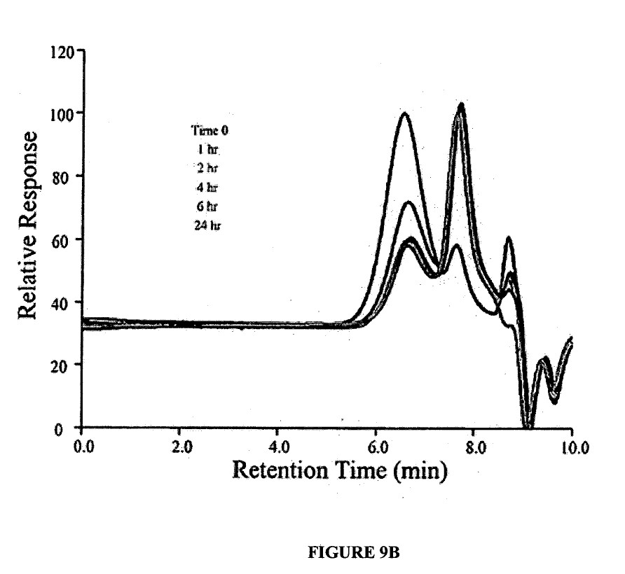

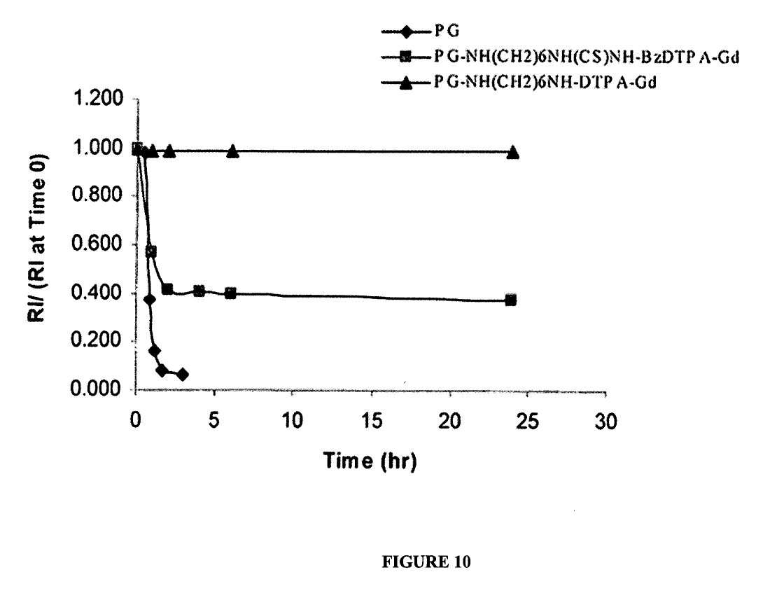

FIG. 9 shows the GPC chromatograms of PG (Mn 17,500) and PG-Hex-BzDTPA-Gd (Mn 21,950). FIG. 10 compares the kinetics of polymer degradation based on the changes in polymer peak height for PG (Mn 17,500), PG-Hex-BzDTPA-Gd (Mn 21,950), and PG-Hex-DTPA-Gd (Mn 222,800). Both PG and PG-Hex-BzDTPA-Gd were degraded at a similar rate during the first 2 h of incubation. However, PG continued to degrade and the polymer was almost completely degraded in 100 min (FIG. 9A and FIG. 10). The degradation rate of PG-Hex-BzDTPA-Gd was much reduced beyond 2 h, possibly as a result of the inability of some break-down products that contained densely packed NH(CH₂)₆NH(CS)NH-BzDTPA-Gd groups on the polymer side chains (FIG. 9B and FIG. 10). These species could not be easily degrade because the bulky side chain groups would prevent the enzyme from approaching the polymers. The degradation of heavily crosslinked PG-Hex-Gd was not observed over a period of 24 h (FIG. 10).

### Example 13

### Synthesis of PG-BzDTPA-Gd

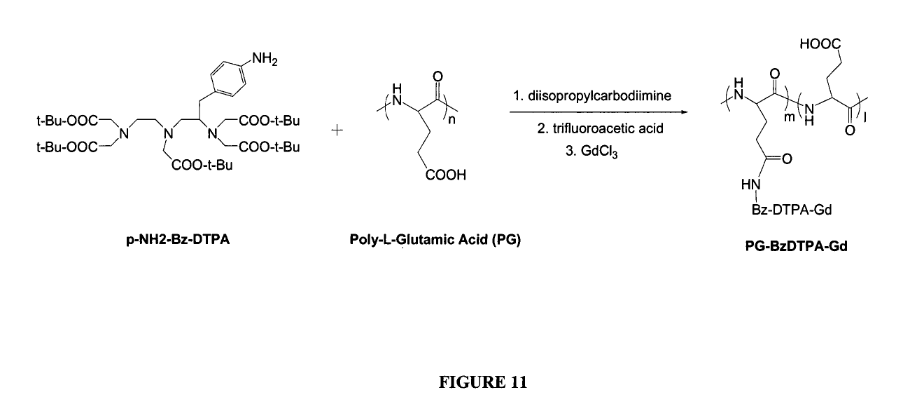

FIG. 11 shows the synthetic scheme for PG-BzDTPA-Gd. Poly-L-glutamic acid (130 mg, 1.0 mmoles of carboxylic unit) and p-aminobenzyl-diethylenetriaminepenta(acetic acid-t-butyl ester) (281 mg, 0.36 mmoles) were dissolved in 5 ml of anhydrous DMF. 1,3-Diisopropylcarbodiimide (52 mg, 0.4 mmoles), 0.16 ml of pyridine and trace amount of 4-dimethylaminopyridine were added. The mixture was stirred at 4° C. overnight. The solvent was removed under vacuum. The residue was dissolved in 2 ml of trifluoroacetic acid in ice bath and stirred at 4° C. overnight. Excess of TFA was removed under vacuum and 10 ml of 1M NaHCO₃ was added in ice bath. The pH of the solution was brought up to 7.5 with 1 M NaOH and dialyzed against PBS and water using dialysis tubing (MWCO 10,000). The resulting solution was filtered through 0.2 μm membrane and lyophilized leaving 230 mg sponge-like powder. Yield was 55%. In the conjugate, 55 of 278 glutamic acid residues were attached with benzyl-DTPA, determined by measuring UV absorbance of benzyl-DTPA at 248 nm.

110 mg of PG-BzDTPA was dissolved in 10 ml of 0.1M sodium acetate (pH5.5) and 0.37 ml of 100 mg/ml GdCl₃ solution in 0.1M sodium acetate was added in six portions. The mixture was checked with indicator PAR (0.2 mM) to be sure only slightly excess of gadolinium was added. (The yellow PAR solution turned red in the presence of trace amount of gadolinium). The reaction solution was dialyzed against water with dialysis tubing (MWCO 10,000) until no free gadolinium was detected in the dialysis water. 100 mg product was given after lyophilization. Yield was 81%. The molecular weight of gadolinium conjugate is about 101,200 as measured by GPC and calculated with Viscotek TriSEC GPC software. The compound contains 12.3% (w/w) of gadolinium as determined by elemental analysis.

The physicochemical properties of PG-BzDTPA-Gd are summarized in Table 4.

TABLE 4
PHYSICOCHEMICAL PROPERTIES OF PG-BZDTPA-GD

| Property | PG-BZDTPA-Gd |
|---|---|
| Number-average MW | |
| Calculated | 70,400 |
| Measured | 101,200 |
| Polydispersity | 1.23 |
| DTPA | 0.20 |
| Gd content, % (w/w) | 12.3 |
| Relaxivity | |
| R₁ (mM⁻¹Sec⁻¹) | 24.8 |
| R₂ (mM⁻¹Sec⁻¹) | 41.0 |

Calculated number-average molecular weight was based upon degree of DTPA substitution and starting PG number-average molecular weight. A number average molecular weight of 42,000 was used for PG (degree of polymerization was 278).

Degree of substitution of DTPA was measured based on UV absorbance of the BzDTPA moiety.

All other measurements and calculations were made in the same manner as in Table 1.

PEG/PG-BzDTPA may be prepared in an analogous fashion from methoxy-PEG-NH₂ and NH₂-Bz-DTPA-penta(t-butyl ester).

### Example 14

### Biodegredation of PG-BzDTPA-Gd

PG-BzDTPA-Gd was dissolved in PBS buffer (pH 5) at a concentration of 5.3 mg/ml. Cathepsin B was added to the solution to a final concentration of 10 units/ml. The degradation was monitored at different time intervals using a gel permeation chromatography (GPC) system equipped with an TSK G3000PW column (TosoHaas), a Waters 515 HPLC pump, and a Viscotek E-Z^{pro} triple detector array. The system was eluted with PBS buffer containing 0.1% LiBr (pH 7.4) at a flow rate of 1 ml/min. Both PG-Hex-BzDTPA-Gd and PG-BzDTPA were degraded when incubated with cathepsin B at room temperature. Degradation of both polymers was completed in 4 hr.

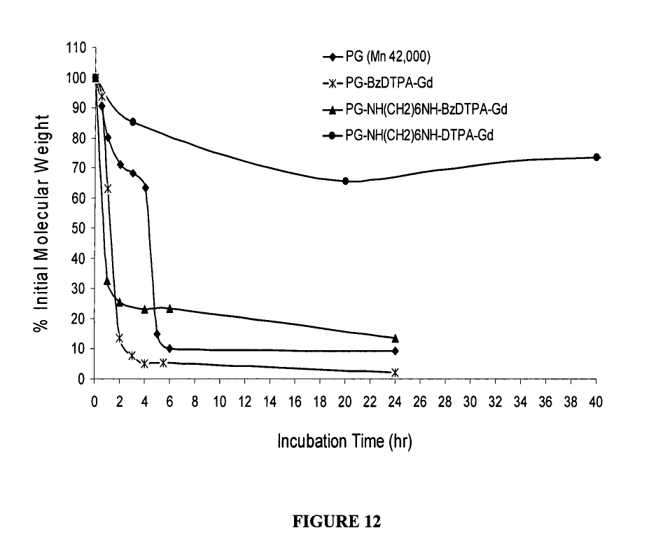

FIG. 12 compares the kinetics of polymer degradation based on the changes in polymer molecular weight for PG (Mn 42,000), PG-BzDTPA-Gd (Mn 101,200), PG-Hex-BzDTPA-Gd (Mn 21,900), and PG-Hex-DTPA-Gd (Mn 222,800). PG, PG-BzDTPA-Gd, and (PG-Hex-BzDTPA-Gd) were rapidly degraded in the presence of Cathepsin B. The degradation of ultrahigh molecular weight, crosslinked PG-NH(CH₂)₆NH-DTPA-Gd was not observed over a period of 24 h.

### Example 15

### Commercial-Scale Production of PG-DTPA-Gd Complexes

Although the above examples focus on laboratory-scale production of PG-DTPA-Gd complexes, they are readily scalable to commercial-scale production. The basic chemical reactions and purifications described may be readily performed on any scale. Diagnostic portions of the examples may be performed on small samples of production lots and they may be used to determine standard amounts to be added during production.

In an exemplary embodiment, compositions of the present invention may be prepared for commercial distribution by lyophilization. Lyophilized samples are preferably stored at 4° C.

### Example 16

### Detection of Therapy-Induced Necrosis

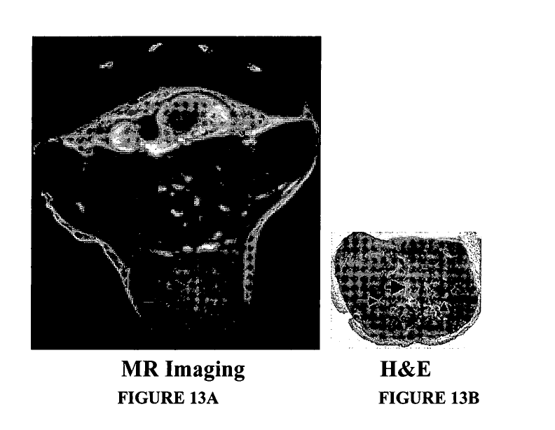

PG-Bz-DTPA-Gd was used to image therapy-induced tumor necrosis in mice. As FIG. 13 shows, upon injection of PG-Bz-DTPA-Gd into a mouse bearing tumors treated with an anti-cancer agent, the central zone of the tumor was significantly enhanced. (FIG. 13A) A macroscopic view of the tumor sectioned in the same plane of the MRI showed areas of necrosis that closely resemble the areas of enhanced signal in the MRI image (FIG. 13B).

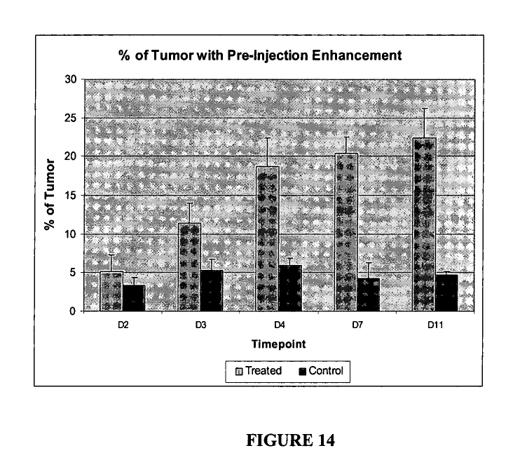

While the percentage of necrotic area did not change over the time period during which MRI scans occurred, the percentage of necrotic area increased steadily over a period of 11 days after treatment. These data suggest that MRI coupled with PG-Bz-DTPA-Gd can be used to monitor the early responses to anticancer therapy. (FIG. 14)

It is likely that the reason for selective accumulation of PG-Bz-DTPA-Gd in the necrotic areas of the tumors is because it is selectively degraded by cathepsin B, an enzyme implicated in tumor invasion and metastasis, arthritis, atherosclerosis, etc., to oligomeric species in the viable region of the tumor. The oligomeric species would then be readily transported to the neighboring necrotic tissues, where they are trapped owing to their interaction with intracellular components of necrotic cells. This novel mechanism would result in markedly increased signal intensity in necrotic tissue and marked contrast between viable and necrotic tissue regions in the late postinjection phase as a result of the following three phenomena: 1) increased tumor uptake of the polymeric contrast agent in the initial postinjection phase, due to the enhanced permeability and retention effect; 2) selective degradation of the contrast agent in the region of viable tumor and subsequent clearance of the degradation products, leading to decreased signal in the viable tumor region; and 3) selective uptake of the oligomeric degradation products in neighboring necrotic tissues within the tumor.

The use of a polymeric imaging probe affords high Gd payload and high relativity. Of the three processes, the action of the enzyme on the imaging probe allows continuous supply of enzyme-catalyzed products to the neighboring target sites. This combination of these three phenomena is herein referred to as the enzyme-mediated signal amplification and neighboring-tissue trapping (EMSANTT) effect.

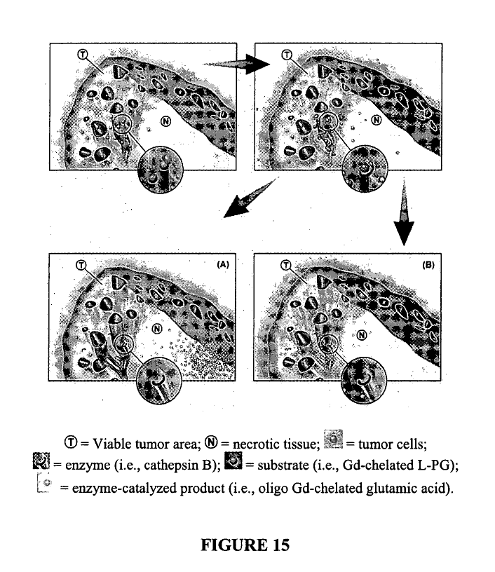

FIG. 15 depicts how the EMSANTT effect might lead to enhanced detection sensitivity. By taking advantage of the high spatial resolution of MRI, it will be possible to design MRI contrast agents that may be used to detect specific enzymatic action and delineate necrotic regions.

Thus, MRI with PG-Bz-DTPA-Gd, or other imaging contrast agents designed with the same concept, such as other contrast agents of the present invention, may be used to simultaneously provide improved detection of necrotic tissues and measure enzyme activity in various diseases. In addition to monitoring responses to anti-cancer therapies, other disease conditions that are associated with necrotic cell death, such as acute myocardial infarction, might also be imaged using PG-Bz-DTPA-Gd or other imaging contrast agents developed with the same principle.

### Example 17

### Mapping of Lymphatic Drain Using PG-DTPA-Gd

Given the present medical need, the suitability of PG-DTPG-Gd was evaluated for rapid identification of sentinal lymph nodes in a small animal model of head and neck squamous carcinoma.

Normal CD1 mice were anesthetized (5% isoflurane in oxygen for induction, 1%-1.5% isoflurane in oxygen for maintenance) and placed head first and prone in a 35 mm linear birdcage-style volume resonator on a 4.7T Biospec scanner (Bruker Biospin, Billerica, Mass.).

Axial, sagittal, and coronal T₁-weighted (TE/TR 9 ms/1000 ms) and T₂-weighted RARE (TE_{eff}/TR 65 ms/4500 ms) baseline images of the head and neck were acquired. Animals were removed from the magnet and varying concentrations of PG-DTPA-Gd in buffered saline were injected into the tongue. Animals were then replaced to approximately the same position, and a set of axial, sagittal, and coronal T₂-weighted images were repeated. Thereafter, axial and coronal T₁-weighted images were repeated, every 12.8 minutes for over 1 hour and 15 minutes.

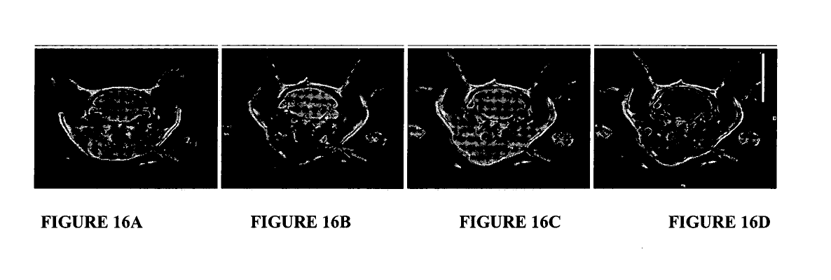

Lymph nodes did not have sufficient contrast with neighboring tissue to be identified in T₁-weighted acquisitions, but could be identified in T₂-weighted images. Regional nodes were enhanced and were visible in T₁-weighted images within 10 minutes of contrast injection, and enhancement remained persistent beyond approximately 90 minutes after injection. FIG. 16 illustrates these results with enhancement of the deep cervical lymph node following injection of 5 mmol of PG-DTPA-Gd in 25 μL of buffered saline.

Thus PG-DTPA-Gd successfully enabled visualization of lymphatic flow from tongue to regional lymph nodes using a minimally invasive imaging procedure requiring no ionizing radiation. Similar success may be expected using other imaging contrast agents of the present invention.

Although the present invention and its advantages have been described in detail, it should be understood that various changes, substitutions and alternations can be made herein without departing from the spirit and scope of the invention as defined by the following claims.

1. A PG polymer complex comprising:
a PG polymer;
at least one DTPA molecule covalently bound to the side chain carboxyl group of the PG polymer; and
at least one Gd, Mn or iron oxide ion chelated to the DTPA.

2. The complex of claim 1, wherein the number average molecular weight of the PG polymer complex is between approximately 10,000 and 500,000.

3. The complex of claim 1, wherein the DTPA molecule is covalently bound to the PG polymer through a linker.

4. The complex of claim 3, wherein the linker comprises a hexene diamine.

5. The complex of claim 3, wherein the linker comprises a PEG polymer of the formula —(CH₂CH₂O)ₙ— where n=1 to 40

6. The complex of claim 1, further comprising at least one PEG polymer covalently bound to the side chain carboxyl groups of the PG polymer.

7. The complex of claim 1, further comprising a targeting molecule covalently attached to the complex.

8. The complex of claim 1, further comprising the complex operable in obtaining an MRI image of a vascular leakage in a patient when administered to the patient.

9. The complex of claim 1, further comprising the complex operable in obtaining an MRI image of inflammation in a patient when administered to the patient.

10. The complex of claim 1, further comprising the complex operable in obtaining an MRI image of angiogensis in a patient when administered to the patient.

11. The complex of claim 1, further comprising the complex operable in obtaining an MRI image of cancer metastases in a patient when administered to the patient.

12. The complex of claim 1, further comprising the complex operable in obtaining an MRI image of a vasculature occlusion in a patient when administered to the patient.

13. The complex of claim 1, further comprising the complex operable in obtaining an MRI image of necrotic tumor tissue in a patient when administered to the patient.

14. The complex of claim 1, further comprising the complex operable in obtaining an MRI image of a lymph node in a patient when administered to the patient.

15. An MRI contrast agent comprising a molecule of the formula:

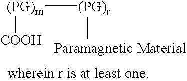

16. The MRI contrast agent of claim 15, further comprising a molecule of the formula: 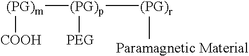
wherein p is at least one and r is at least one.

17. The MRI contrast agent of claim 15, further comprising PG-BzDTPA-Gd.

18. A method of making a PG polymer complex comprising:
modifying at least one side chain carboxyl groups of a PG polymer;
attaching at least one chelating agent molecule to a modified side chain carboxyl group; and
chelating a paramagnetic material.

19. The method of claim 18, wherein at least 5% of all available side chain carboxyl groups of the PG polymer are modified.

20. The method of claim 18, wherein a chelating agent molecule is attached to at least 5% of all available modified side chain carboxyl groups.

21. The method of claim 18, wherein the chelating agent comprises DTPA.

22. The method of claim 18, wherein the paramagnetic material comprises Gd, Mn or iron oxide.

23. The method of claim 18, further comprising attaching at least one PEG polymer to a modified side chain carboxyl group.

24. The method of claim 18, further comprising attaching at least one targeting molecule to the PG polymer, wherein the targeting molecule is selected from the group consisting of: antibodies, homing ligands, ligands that home to growth factor receptors, cytokines, integrins and combinations thereof.
